(19) United States
(12) Patent Application Publication
(10) Pub. No.: US 2002/0028258 A1
(43) Pub. Date: Mar. 7, 2002

(54) Immunostimulant compositions and associated methods

(75) Inventors: Lester A. Mitscher, Lawrence, KS (US); Segaran P. Pillai, Miami, FL (US); Raymond Cooper, Mountain View, CA (US)

Correspondence Address: THORPE NORTH WESTERN, 8180 SOUTH 700 EAST, SUITE 200, P.O. BOX 1219, SANDY, UT, 84070, US

(73) Assignee: Pharmanex, LLC

(21) Appl. No.: 09/852,132

(22) Filed: May 9, 2001

Related U.S. Application Data
(60) Provisional application No. 60/203,028, filed on May 9, 2000.

(51) Int. Cl. A61K 35/78; A61K 31/715

(52) U.S. Cl. 424/737; 514/54

(57) ABSTRACT

The present invention provides immunostimulant compositions and methods for the preparation and use thereof which contain a therapeutically effective amount of an active polysaccharide containing Echinacea extract. In one aspect, the method for making an immunostimulant composition may include the steps of: providing an Echinacea source, extracting an active polysaccharide from the Echinacea source into an aqueous solution, precipitating a water precipitate containing the active polysaccharide from the aqueous solution; and recovering the water precipitate.

### PRIORITY DATA

This patent application claims priority to U.S. Provisional Application No. 60/203,028, filed May 9, 2000, which is incorporated herein by reference in its entirety.

### THE FIELD OF THE INVENTION

The present invention relates generally to immunostimulant compounds and methods for making such compounds. More particularly, it concerns an immunostimulant composition having a high concentration of an active polysaccharide and other active agents which are extracted from Echinacea.

### BACKGROUND OF THE INVENTION

Echinacea has been used as a remedy for a variety of ailments in traditional folk medicinal, and has been moderately used in modern medicine since about 1870. While a variety of Echinacea species exists, the species which have been most widely used as a medicinal treatment, or health supplement have been the species Echinacea purpurea, Echinacea pallida, and Echinacea angustafolia.

Echinacea has been administered most widely for its apparent ability to combat various afflictions such as influenza, colds, and the like. A variety of ingredients has been attributed to Echinacea's disease fighting activity. Such purported active ingredients include caffeic acids, flavanoids, polyacetylenes, alkylamides, alkaloids, and polysaccharides.

The physiological effects of many such ingredients have been hypothesized. However, as of yet none of Echinacea's ingredients has been conclusively linked to a specific activity that accounts for its illness combating properties.

Because the exact nature and function of Echinacea's ability to combat illness has remained a mystery, it has been impossible to target, remove, and purify the specific active agents which are primarily responsible. Therefore, most Echinacea dosages have simply been an amount of the raw plant which is harvested, dried, and ground into powder. Such dosages are inefficient, and may include substances which are not desirable for consumption, or which may reduce the efficacy of the active agents. Further, such dosages are inconsistent in the amount of active agents which are delivered. Additionally, such dosages do not maximize the therapeutically active ingredients while minimizing or eliminating undesirable or superfluous ingredients.

Therefore, the determination of those Echinacea ingredients that impart a therapeutic effect in combating illness continues to be the focus of ongoing research efforts. Further, determining the specific physiological effect of various Echinacea active ingredients continues to be the subject of current research efforts.

### SUMMARY OF THE INVENTION

Accordingly, the present invention provides a method of preparing an immunostimulant composition includes the steps of: providing an Echinacea source; extracting an active polysaccharide from the Echinacea source into an aqueous solution; precipitating a water precipitate containing the active polysaccharide from the aqueous solution; and reclaiming, or recovering, the water precipitate containing the active polysaccharide. Additional steps which may be performed as part of such a method prior to the step of precipitating include without limitation, the steps of: removing the Echinacea source from the aqueous solution; adding an organic solvent to the aqueous solution to remove organic materials therefrom; and reducing the solution to a lower volume. In one aspect, the aqueous solution may have a temperature from about 10° C. to about 45° C. In another aspect, the aqueous solution may have a pH of from about 5 to about 7.

The formation of the water precipitate may be accomplished by a variety of methods known to those skilled in the art. In one aspect, the step of precipitating may be accomplished by adding a precipitant to the aqueous solution to form the precipitate containing the active polysaccharide. A variety of acceptable precipitants may be used, and will be within the knowledge of the skilled artesian. However, in one aspect, the precipitant may be an alcohol. Various types of alcohol may be used as the precipitant, including lower alkanols having from 1-4 carbon atoms. In one aspect, the lower alkanol may be ethanol.

In addition to the above-recited steps, the present method may achieve a greater purification of active polysaccharides by further including the step of: dividing the recovered water precipitate into separate components according to size. Those of ordinary skill in the art will recognize a variety of ways in which such a separation may be accomplished. However, in one aspect, the step of dividing may be accomplished utilizing size exclusion chromatography.

The Echinacea source used in the present invention may include roots, aerial parts, such as leaves and stem, or a combination thereof. Further, a wide variety of Echinacea species may be used. In one aspect, the Echinacea source may be a species selected from the group consisting of: Echinacea purpurea, Echinacea angustifolia, Echinacea pallida, and mixtures thereof. In another aspect, the Echinacea source may be Echinacea purpurea. In a further aspect, the Echinacea source may be Echinacea angustifolia. In yet another aspect, the Echinacea source may be Echinacea pallida.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### BRIEF DESCRIPTION OF DRAWINGS

FIG. 1 is a graphical representation of the dosage dependent stimulation in immune cell activity in response to receiving an active polysaccharide containing Echinacea extract. Particularly, the immune cells have a CD4+surface antigen, and have been treated with an extract of E. purpurea flowerlets prepared in accordance with one embodiment of the present invention.

FIG. 2 is a graphical representation of the dosage dependent stimulation in immune cell activity in response to receiving an active polysaccharide containing Echinacea extract. Particularly, the immune cells have a CD8+ surface antigen, and have been treated with an extract of E. purpurea flowerlets prepared in accordance with one embodiment of the present invention.

FIG. 3 is a graphical representation of the dosage dependent stimulation in immune cell activity in response to receiving an active polysaccharide containing Echinacea extract. Particularly, the immune cells have a CD19+ surface antigen, and have been treated with an extract of E. purpurea flowerlets prepared in accordance with one embodiment of the present invention.

FIG. 4 is a graphical representation of the dosage dependent stimulation in immune cell activity in response to receiving an active polysaccharide containing Echinacea extract. Particularly, the immune cells have a CD56+ surface antigen, and have been treated with an extract of E. purpurea flowerlets prepared in accordance with one embodiment of the present invention.

FIG. 5 is a chromatograph of an Echinacea composition prepared in accordance with one embodiment of the present invention, which is undergoing isolation of active polysaccharides using a size exclusion chromatography technique.

### DETAILED DESCRIPTION

Definitions

Before the present formulations and methods are disclosed and described, it is to be understood that the present invention is not limited to the particular process steps and materials disclosed herein, but is extended to equivalents thereof as would be recognized by those ordinarily skilled in the relevant arts. It should also be understood that terminology employed herein is used for the purpose of describing particular embodiments only and is not intended to be limiting.

In describing and claiming the present invention, the following terminology will be used.

The singular forms “a,” and, “the” include plural referents unless the context clearly dictates otherwise. Thus, for example, reference to an Echinacea extract containing “a polysaccharide component” includes one or more polysaccharide components, reference to “an immune system component” includes reference to one or more of such components, and reference to “the dosage form” includes reference to one or more dosage forms.

In describing and claiming the present invention, the following terminology will be used in accordance with the definitions set forth below.

As used herein, “Echinacea,” “Echinacea plant,” “Echinacea agent,” and “Echinacea extract,” may be used interchangeably herein, and refer to an extract made from the roots, or aerial parts, such as leaves, stems, flowers, or any other physiological portion of any Echinacea plant species, including all strains and hybrids, or of plants significantly related to thereto, grown anywhere in the world including blends, mixtures, and combinations of such strains and relatives. Examples of individual Echinacea species include without limitation, Echinacea angustifolia [Braumeria angustifolia], Echinacea atrorubens [Rudbeckia atrorubens], Echinacea laevigata [B. laevigata], Echinacea pallida [R. pallida, B. pallida], Echinacea paradoxa [B. paradoxa], Echinacea purpurea [R. purpurea, R. hispida, R. serotina, E. speciosa, E. intermedia], Echinacea simulata [E. speciosa], Echinacea sanguinea, and Echinacea tennesseensis [B. tennesseensis].

The terms “formulation” and “composition” may be used interchangeably herein.

As used herein, “immune cell” refers to a cell which plays an active role in the immune system of a healthy body. The scope, type, and specific function of such cells is well known to those of ordinary skill in the art. Immune cells are divided into two general types, phagocytes and lymphocytes. Phagocytes include neutrophils, monocytes, and macrophages. Lymphocytes include natural killer cells, T-cells, and B-cells. A general discussion of each of the above-recited immune cells and each of their respective roles may be found in The Merck Manual, pg. 1002-1022 (17^{th} ed. 1999).

As used herein, “polysaccharide” refers to a compound containing a combination of nine or more monosaccharides which are linked together by glycosidic bonds. Further, an “active polysaccharide” refers to a polysaccharide which is capable of stimulating immune cell activity by interacting with surface receptors on an immune cell.

As used herein, “immunostimulant”, and “immunostimulant agent” may be used interchangeably, and refer to an agent, such as an active polysaccharide, which is capable of stimulating immune cell activity.

As used herein, “ED” refers to an effective dose, and “ED50” or “median effective dose” refer to the median, or average dose which produces a desired effect in about 50% of subjects tested.

As used herein, “an alcohol” refers to an alcohol solution containing a concentration of alcohol that is sufficient to cause precipitation of a water precipitate containing active polysaccharides when added to an aqueous polysaccharide containing solution. Further, “lower alkanol” refers to an alcohol containing from 1 to 6 carbon atoms. Examples of lower alkanols include without limitation, methanol, ethanol, propanol, isopropanol, etc. Use of the term “alcohol” without any designation of the number of carbon atoms shall be considered to be ethanol.

As used herein, an “effective amount,” and “sufficient amount” may be used interchangeably and refer to an amount of an ingredient which, when included in a composition, is sufficient to achieve an intended compositional or physiological effect. For example, a “sufficient amount” of a solvent would be the minimum amount needed to dissolve a target substance to a selected degree. Further, a “therapeutically effective amount” refers to an amount of a polysaccharide which is sufficient to achieve a desired immunostimulant effect. The determination of an effective amount is well within the ordinary skill in the art of pharmaceutical, neutraceutical, herbaceutical, cosmetic, and medical sciences. See, for example, Meiner and Tonascia, “Clinical Trials: Design, Conduct, and Analysis,” Monographs in Epidemiology and Biostatistics, Vol. 8 (1986), incorporated by reference in its entirety.

As used herein, “active agent” refers to an agent contained in an Echinacea extract which imparts, or is capable of imparting or inducing a measurable physiological effect when administered to the body. Examples of active agents include without limitation, polysaccharides, caffeic acids, flavanoids, cicoric acid, ecinacoside, polyacetylenes, alkylamides, alkaloids, and constituents thereof.

As used herein, “organic solvent” refers to any water immiscible organic solvent, for example without limitation, ethyl acetate and chloroform.

As used herein, “precipitant” refers to a substance that may be added to an aqueous solution to cause the formation of a precipitate. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a concentration range of “about 0.1% w/w to about 25% w/w” should be interpreted to include not only the explicitly recited concentration of about 0.1% to about 25% w/w, but also include individual concentrations and the sub-ranges within the indicated range. Thus, included in this numerical range are individual concentrations such as 2% w/w, 5% w/w, and 6% w/w, and sub-ranges such as from 1% w/w to 3% w/w, from 2% w/w to 6% w/w, from 8% w/w to 18% w/w, from 5% w/w to 20% w/w, etc. The same principle applies to ranges reciting only one numerical value.

Similarly, a range recited as “less than about 5.8% w/w” should be interpreted to include all of the values and ranges as elaborated above for the range of “from about 0.1% w/w to about 25% w/w.” Furthermore, such an interpretation should apply regardless of the breadth of the range or the characteristics being described.

Invention

Applicants have discovered that active polysaccharides, particularly those with a molecular weight in the range of from about 30,000 to 70,000 are capable of stimulating immune cell activity. In one aspect, such stimulation may be accomplished by polysaccharide interaction with immune cell surface receptors which activate the cell and may trigger a cascading release of cytokines and/or chemokines. Once released, the cytokines and/or chemokines may further activate or direct the polysaccharide activated cell, as well as other immune cells.

The present invention provides immunostimulant compositions and methods for producing immunostimulant compositions. Further, standardized immunostimulant dosages and methods of stimulating immune cell activity are encompassed by the present invention.

Generally, the method of producing an immunostimulant composition includes the steps of: providing an Echinacea source, extracting an active polysaccharide from the Echinacea source into an aqueous solution, precipitating a water precipitate from the aqueous solution containing the active polysaccharide, and recovering the precipitate.

A wide variety of Echinacea species may be used in connection with the present invention. By way of example without limitation, acceptable Echinacea species include: Echinacea angustifolia [Braumeria angustifolia], Echinacea atrorubens [Rudbeckia atrorubens], Echinacea laevigata [B. laevigata], Echinacea pallida [R. pallida, B. pallida], Echinacea paradoxa [B. paradoxa], Echinacea purpurea [R. purpurea, R. hispida, R. serotina, E. speciosa, E. intermedia], Echinacea simulata [E. speciosa], Echinacea sanguinea, and Echinacea tennesseensis [B. tennesseensis]. In one aspect, the Echinacea source may be Echinacea purpurea. In another aspect, the Echinacea source may be Echinacea angustifolia. In yet another aspect, the Echinacea source may be Echinacea pallida.

The entire Echinacea plant may be used as the Echinacea source, or a portion thereof only. Aerial parts, such as the stem, leaves, etc. may be utilized with the present invention as well as the roots of the Echinacea plant. Additionally the manner in which the Echinacea source material is introduced into the aqueous solution may vary. In one aspect, the Echinacea source may be in a dried powdered form. In another aspect, the Echinacea source may be in a ground fresh form. In yet another aspect, the Echinacea source may be a fresh juice in a lyophilized form. In yet another aspect, the Echinacea source may be in a fresh blended liquid form.

Pretreatment of the Echinacea source with various chemicals may also be performed prior to introducing it into the aqueous solution. In one aspect, the Echinacea source may be pretreated using one or more alcohols. Such pretreatments are described in further detail in the examples listed below.

Once introduced into the aqueous solution, active polysaccharides and other active ingredients are removed from the Echinacea source due in large measure to their respective solubilities. Additionally, active agent removal from the Echinacea source may be facilitated by the percolation, or movement of the aqueous solution through the Echinacea source. Movement of the aqueous solution may be provided in a variety of ways, such as gravity producing a downward flow of aqueous solution through the Echinacea source, convection forces created by heat applied to the solution, mechanical agitation, such as by stirring, shaking, sonicating, etc., and by chemical forces, such as effervescent action caused by the interaction of effervescent chemical ingredients. Those of ordinary skill in the art will readily recognize the full spectrum of methods for customizing or optimizing active agent extraction so as to achieve a specifically desired result.

The active polysaccharides which are extracted from the Echinacea source and utilized in the present invention includes a wide variety of specific polysaccharide types. In one aspect, the polysaccharide may have a molecular weight of from about 30,000 to about 70,000. In another aspect, a single type of polysaccharide may be isolated and used. In a further aspect, a mixture of polysaccharide types may be used. Those ordinarily skilled in the art will appreciate a variety of methods for isolating and purifying specific polysaccharides.

Contrary to conventional teachings, applicants have found that the fraction of Echinacea with the greatest amount of immunostimulant activity is the water soluble fraction. To this end, the precipitate formed by the present invention is a water precipitate and will contain a rich active polysaccharide content. The water fraction may additionally contain other active agents as discussed herein. The precipitation of the water precipitate may be accomplished by a variety of specific methods as will be readily recognized by those skilled in the art, and will depend largely on the composition of the aqueous solution.

In one aspect, the aqueous solution may have a temperature of from about 10° C. to about 45° C., and a pH of from about 5 to about 7. In another aspect, the solution may consist entirely of water, such as deionized water. In yet another aspect, the solution may include effective amounts of other ingredients to aid in the active agent extraction.

In one aspect, the polysaccharide containing precipitate, may be formed using an alcohol as the precipitant. The alcohol utilized will generally be a lower alkanol having from about 1 to about 4 carbons. In one aspect, the alcohol may be ethanol. Ethanol may be desirable, as residual amounts which may become admixed with the recovered precipitate generally do not pose a significant health risk. Other lower alkanols may be utilized, such as isopropanol. However, in such an event it may be desirable to remove any residual amounts thereof utilizing a moderate amount of heat, up to about 90° C. or less, or a consumable acid such as acetic acid, etc. Other methods of removing residual alcohol amounts without degrading the active agents in the solution will be readily apparent to those skilled in the art.

The volume of alcohol or water required to form the polysaccharide-containing water precipitate may be readily determined by one of ordinary skill in the art, and may be varied in order to achieve a specific result. However, in one aspect, the volume of alcohol may be about 5 times larger than the volume of water in the aqueous solution.

In one aspect, the Echinacea source material will be removed from the aqueous solution prior to forming the water precipitate. A variety of methods for removing such material will be readily recognized by those skilled in the art. In one aspect, the material may be removed via straining with a cloth, or other sieve having pores of appropriately small size.

Additional steps may be performed while the active polysaccharides are in the aqueous solution, and before the formation of a precipitate. In one aspect, the aqueous solution may be reduced in volume or concentrated. In another aspect, an organic solvent may be added to the aqueous solution in order to dissolve any organic matter that may be somewhat toxic. In one aspect, the organic solvent may be a water immiscible organic solvent. In another aspect, the water immiscible organic solvent may be ethyl acetate. In yet another aspect, the water immiscible organic solvent may be chloroform.

Reclamation of the water precipitate containing the active polysaccharides may be accomplished by a variety of methods known to those skilled in the art. Physical separation techniques are widely known and quite numerous. In connection with the present invention, the only limitation placed on the use of such techniques is that they may not degrade, destroy, or inactivate the active polysaccharides contained in the precipitate.

The active polysaccharide fraction of the recovered water precipitate may be further isolated using a variety of techniques known to those skilled in the art. One example of a technique which may be used is size exclusion chromatography. Details of specific size exclusion chromatography techniques an parameters are found in Example 9 below.

The immunostimulant compositions of the present invention includes high concentrations of active polysaccharides which are obtained from Echinacea. In one aspect, the active polysaccharide concentration may be from about 5% w/w to about 90% w/w of the immunostimulant composition. In another aspect, the active polysaccharide concentration may be greater than about 75% w/w of the immunostimulant composition. In a further aspect, the active polysaccharide concentration may be greater than about 85% w/w of the immunostimulant composition. In yet another aspect, the active polysaccharide concentration may be greater than about 90% w/w of the immunostimulant composition. In a further aspect, the active polysaccharide concentration may be greater than about 95% w/w of the immunostimulant composition.

Immunostimulant compositions containing such amounts of polysaccharides have shown to be capable of greatly increasing immune cell activity. As shown in the examples set forth below, the compositions of the present invention may stimulate immune cells of various type in an amount of from about 4.38% to about 80.30% depending on the type and amount of Echinacea extract used and the type of specific immune cell being quantified.

In addition to active polysaccharides, other ingredients may be included in the immunostimulant composition of the present invention. A wide range of other types of ingredients may be added which provide further immune cells stimulation, or augment the body's immune system in other ways.

As will be recognized by those skilled in the art, a wide variety of positive health benefit imparting ingredients may be selected from natural sources, such as herbal and botanical extracts, as well as other sources, such as medicinal compounds and be added as desired in order to achieve a specific therapeutic result. Such additions may be made by the skilled artesian without undue experimentation.

Generally, herbal and botanical extracts are made from all kinds of herb and botanic sources and formulated based on their therapeutic function. For example, anti-flu, bone/joint, brain function, cardiovascular, circulatory, diet, depression, digestion, energy, eye vision, general health, immune system, liver, men's health respiratory, rest, urinary tract, women's health, etc. In one aspect, herbal and botanical extracts for inclusion in the present formulation can be selected from, but not limited to, Ginseng, Ginko Biloba, Dong Quai, Hawthorn berry, St. John's Wort, Saw Palmetto, Kava Kava, Rose Hips, Licorice Root, Grape seed, Chammomile, Sea Buckthorn, Aloe Vera, Cinnamon Bark, Cordyceps, Ho Shou Wu, Dandelion, Gynostemma, mushroom, Notginseng, Dan Shen, and mixtures thereof may be included.

In one aspect, vitamins either water soluble or oil soluble may be added. Water soluble vitamins specifically contemplated by the present invention include, but are not limited to: vitamin B₁, B₂, B₃, B5, B₆, B₁₂, B₁₃, B₁₅, B₁₇, biotin, choline, folic acid, inositol, para-aminobenzoic acid (PABA), vitamin C, and vitamin P. Additionally, oil soluble vitamins include, but are not limited to: vitamin A, vitamin D, vitamin E, and vitamin K.

Other health imparting substances which may be combined with the desired Echinacea extract in the formulation of the present invention include amino acids, ionic minerals, and naturally occurring anti-oxidants. The amino acids may include without limitation: alanine, arginine, carnitine, gamma-aminobutyric acid (GABA), glutamine, glycine, histidine, lysine, methionine, N-acetyl cysteine, ornithine, phenylalanine, taurine, tyrosine, and valine, but are not limited thereto. Additionally, the ionic minerals contemplated by the present invention for inclusion in an embodiment of the formulation include both anions and cations. For example, calcium, zinc, copper, magnesium, manganese, phosphorous, fluoride, etc. Finally, the naturally occurring anti-oxidants contemplated for the formulation of the present invention include: grape seed, beta-carotene, and co-enzyme Q-10, but are not limited thereto.

Because of the highly purified active polysaccharide fraction of the present immunostimulant composition, it is possible to prepare a standardized dosage formulation. A variety of dosage formulation are known to those skilled in the art and include without limitation, oral, transdermal, transmucosal, parenteral, and intravenous dosages. Examples of specifically acceptable oral dosage formulations include without limitation, tablets, capsules, liquid dosage formulations, effervescent preparations, food preparations, and confection or candy dosage forms. Examples of transdermal or transmucosal formulations include without limitation, transdermal patches, such as adhesive matrix and liquid reservoir patches, oral transmucosal tablets, lozenges, ointments, pastes, lacquers, etc. Injectable formulations include without limitation, bolus injection dose, intravenous injection and drip formulations.

The present invention additionally encompasses methods for stimulating immune cells. Such methods include the steps of: administering to a subject a composition as described herein, or administering a composition prepared by a method as described herein, which contains a therapeutically effective amount of an active polysaccharide containing Echinacea extract.

### EXAMPLES

Several general methods for preparing Echinacea extracts are employed in the examples recited below. Generally, three specific extraction protocols are employed to a variety of Echinacea sources.

Echinacea Extraction Method 1

Twenty grams of dried powdered Echinacea source material which has previous been subjected to ethanol extraction as known in the art, followed by a methanol wash is provided. A first water extraction is performed in 200 ml of deionized water for 4 hours at 37° C. with continuous gyrorotory shaking. The Echinacea source material is removed and a second water extraction is performed in 200 ml of deionized water for 18 hours at 37° C. with continuous gyrorotory shaking. The Echinacea source material is then removed and the water extracts are combined.

Echinacea Extraction Method 2

Twenty grams of dried powdered Echinacea source material is extracted in 200 ml of deionized water for 4 hours at 37° C. with continuous gyrorotory shaking. The Echinacea source material is removed and a second water extraction is performed in 200 ml of deionized water for 18 hours at 37° C. with continuous gyrorotory shaking. The Echinacea source material is then removed and the water extracts are combined.

Echinacea Extraction Method 3

Following the extraction steps of Method 2, a small quantity of ethyl acetate, or other water immiscible organic solvent is added to the water extract in order to solubilize organic impurities contained within the extract.

Echinacea Extraction Method 4

Following the extraction steps of Method 2, the water extract is admixed with an amount of alcohol which is approximately 5 times larger than the volume of the water extract. The mixture may then be agitated and forms a water precipitate. The water precipitate containing the Echinacea active agents is then removed.

Immune Cell Stimulation Assay Method

Each immunostimulation assay uses a Becton Dickinson FACScan flow cytometer to quantify immune cell activity. The specific protocol for measuring such activity utilizes freshly drawn human blood collected in sodium heparin to 200 μL. Echinacea extract is added in a desired concentration after being mixed with 50 μL of phosphate buffer. The blood and Echinacea mixture is vortexed and incubated at 37° C. for 5 hours to allow adequate time for immune cell stimulation.

Positive controls consist of PMA (50 ng/ml), PHA (10 pL/ml), or CD2/CD2R (5 μL/250 μL) and negative controls consist of DMSO (2% in RPMI), RPMI, and cyclosporin A (10 μg/ml). For staining, 20 μL of reagent cocktail (CD4FITC/CD69PE/CD3PerCP (expressed on the helper/inducer T-lymphocyte subset and is the primary receptor for HIV); CD8FITC/CD69PE/CD3PerCP (expressed on the human suppressor/cytotoxic T-lymphocytes and on a subset of natural killer lymphocytes); CD19FITC/CD69PE/CD45PerCP (expressed on B-lymphocytes); CD56FITC/CD69PE/CD45PerCP (expressed on NK lymphocytes), for examples) are used.

Following the cell stimulation period described above, solutions of fractions and constituents are compared quantitatively with the above-recited controls. Next 50 μL of the stimulated blood samples are added to each of the tubes containing the reagent cocktail, vortexed and incubated at room temperature for 30 min. in the dark. Then 450 μL of 1× FACS lysing solution is added to each tube which is vortexed gently and incubated in the dark at room temp. for 20 min. The prepared samples are analyzed in the flow cytometer. CD69 (CD stands for cluster determinant) is the first cytokine expressed by any of these cells following stimulation and is produced on the surface of T-, B- and NK-lymphocytes. Its increase indicates that this cell line is responding by dividing and maturing. Individual cell types are quantitated using appropriate stains and gating as known by those skilled in the art.

### Example 1

Extracts of Echinacea purpurea are prepared according to Echinacea Extraction Methods 1 and 3 and concentrated to a desired level of concentration. The extracts are then tested for immunostimulatory effects using the Immune Cell Stimulation Assay Method. CD4+stimulation results are provided in Table 1.

TABLE 1
Comparison of Immunostimulation of Methods 1 and 3

| | | | | | | | | |
|---|---|---|---|---|---|---|---|---|
| Conc. ug/ml | 25 | 50 | 100 | 200 | 400 | 800 | 1600 | 3200 |

% CD4+ Stimulation

| | | | | | | | | |
|---|---|---|---|---|---|---|---|---|
| Method 1 | 33.54 | 33.59 | 36.39 | 35.99 | 34.63 | 29.27 | 26.70 | 27.91 |
| Method 3 | 25.31 | 34.52 | 36.08 | 39.48 | 34.67 | 32.10 | 28.10 | 26.27 |
| EtOAc | 6.22 | 5.18 | 4.61 | 3.55 | 4.05 | 4.87 | 4.85 | 41.67* |
| Fraction | | | | | | | | |

*Improper gating-measuring toxicity-No activity

### Example 2

Extracts of E. purpurea, E. Angustifolia, and E. pallida are prepared using various plant parts in accordance with Extraction Method 3 and concentrated to a desired level of concentration. The extracts are then tested for immunostimulatory effects using the Immune Cell Stimulation Assay Method. CD4+ stimulation results are provided in Table 2.

TABLE 2
Immunostimulatory effects of various Echinacea
Strains Extracted Using Method 3

| | | |
|---|---|---|
| Controls | | |
| Negative | DMSO (2% in RPMI) | 4.53% |
| | RPMI | 4.70% |
| | CspA (10 ug/ml) | 4.22% |
| Positive | PMA (50 ng/ml) | 99.16% |
| | CD2/CD2R | 24.04% |
| | (5 ul/250 ul rxn) | |
| Experimental | | |

| | | | |
|---|---|---|---|
| Conc. ug/ml | 25 | 50 | 100 |
| E.purpurea | | | |
| Roots | 40.54% | 39.94% | 41.35% |
| Stem/Leaves | 41.02% | 45.00% | 45.99% |
| Flowerlets | 44.79% | 45.57% | 47.15% |
| E. angustifolia | | | |
| Roots | 56.81% | 58.13% | 61.07% |
| Stem/Leaves | 48.61% | 52.05% | 59.84% |
| Flowerlets | 55.68% | 56.41% | 60.45% |
| E. pallida | | | |
| Roots | 46.31% | 50.85% | 52.25% |
| Stem/Leaves | 45.73% | 46.31% | 48.54% |
| Flowerlets | 54.61% | 56.90% | 58.50% |

### Example 3

Extracts of E. purpurea are prepared from various plant parts in accordance with Extraction Method 4 and concentrated to a desired level of concentration. The extracts are then tested for immunostimulatory effects using the Immune Cell Stimulation Assay Method. CD4+ stimulation results are provided in Table 3.

TABLE 3
Immunostimulatory effects of various Echinacea
purpurea Parts Extracted Using Method 4

| | | |
|---|---|---|
| Controls | | |
| Negative | DMSO (2% in RPMI) | 5.94% |
| | RPMI | 7.93% |
| | CspA (10 ug/ml) | 5.58% |
| Positive | PMA (50 ng/ml) | 99.16% |
| | CD2/CD2R | 24.04% |
| | (5 ul/250 ul rxn) | |
| Experimental | | |

| | | | |
|---|---|---|---|
| Conc. ug/ml | 6.25 | 12.5 | 25 |
| E. purpurea | | | |
| Roots | 53.52% | 59.53% | 65.21% |
| Stem | 47.15% | 53.71% | 57.23% |
| Leaves | 55.44% | 57.75% | 58.20% |
| Flowerlets | 57.77% | 57.81% | 65.38% |

### Example 4

Extracts of E. purpurea are prepared from various plant parts in accordance with Extraction Method 4 and concentrated to a desired level of concentration. The extracts are then tested for immunostimulatory effects using the Immune Cell Stimulation Assay Method. CD4+stimulation results are provided in Table 4.

TABLE 4
Immunostimulatory effects of various Echinacea
purpurea Parts Extracted Using Method 4

| | | |
|---|---|---|
| Controls | | |
| Negative | DMSO (2% in RPMI) | 5.83% |
| | RPMI | 6.72% |
| | CspA (10 ug/ml) | 7.25% |
| Positive | PMA (50 ng/ml) | 99.20% |
| | PHA (20 ug/ml) | 16.98% |
| | CD2/CD2R | 47.25% |
| | (5 ul/250 ul rxn) | |
| Experimental | | |

| | | | |
|---|---|---|---|
| Conc. ng/ml | 48.75 | 97.5 | 195 |
| E. purpurea | | | |
| Roots | 13.40% | 17.12% | 20.78% |
| Stem | 8.29% | 10.55% | 10.18% |
| Leaves | 10.58% | 11.56% | 16.35% |
| Flowerlets | 22.15% | 30.36% | 31.92% |

### Example 5

Extracts of Echinacea angustifolia roots are prepared according to the various Echinacea extraction methods recited above, and concentrated to a desired level of concentration. The extracts are then tested for immunostimulatory effects using the Immune Cell Stimulation Assay Method. CD4+ stimulation results are provided in Table 5.

TABLE 5
Comparison of Immunostimulation by E. Augustifolia
Root Extracts Prepared by Various Methods

| | | | | | | | | |
|---|---|---|---|---|---|---|---|---|
| Conc. ug/ml | 1.56 | 3.125 | 6.25 | 12.5 | 25 | 50 | 100 | 200 |

% CD4+ Stimulation

| | | | | | | | | |
|---|---|---|---|---|---|---|---|---|
| Method 1 | 18.15 | 27.42 | 34.90 | 39.33 | 46.31 | 46.02 | 47.40 | 57.85 |
| Method 2 | 42.98 | 45.82 | 46.76 | 50.48 | 54.89 | 57.87 | 61.45 | 65.45 |
| Method 3 | 41.59 | 43.39 | 45.91 | 51.50 | 56.50 | 61.13 | 63.59 | 66.53 |
| Method 4 | 41.58 | 45.17 | 46.58 | 51.66 | 57.83 | 60.46 | 63.15 | 63.14 |
| Method 1 + 4 | 33.17 | 33.80 | 41.49 | 43.41 | 48.58 | 53.31 | 59.03 | 65.26 |

### Example 6

Extracts of Echinacea angustifolia roots are prepared according to the various Echinacea extraction methods recited above, and concentrated to a desired level of concentration. The extracts were then tested for immunostimulatory effects using the Immune Cell Stimulation Assay Method. CD4+ stimulation results are provided in Table 6.

TABLE 6
Comparison of Immunostimulation by E. Angustifolia
Root Extracts Prepared by Various Methods

| | | | | | |
|---|---|---|---|---|---|
| Conc. ng/ml | 48.75 | 97.50 | 195 | 390 | 780 |
| % CD4+ | | | | | |
| Stimulation | | | | | |
| Method 1 | 4.38 | 5.36 | 6.36 | 8.37 | 10.30 |
| Method 2 | 12.02 | 16.56 | 20.15 | 25.46 | 28.03 |
| Method 3 | 17.69 | 18.25 | 20.91 | 26.06 | 28.52 |
| Method 4 | 17.01 | 20.46 | 24.46 | 28.86 | 30.89 |
| Method 1 + 4 | 4.56 | 6.19 | 7.52 | 11.90 | 14.94 |

### Example 7

Extracts of E. angustifolia roots are prepared in accordance with Extraction Method 4 and concentrated to a desired level of concentration. The extracts are then tested for immunostimulatory effects using the Immune Cell Stimulation Assay Method. CD4+, CD8+, CD19+, and CD56+ stimulation results are provided in Table 7.

TABLE 7
Immunostimulatory Effects of Echinacea Angustifolia
Root Extracts Prepared Using Method 4

| | | | |
|---|---|---|---|
| CD4+ | CD8+ | CD19+ | CD56+ |

Controls

| | | | | |
|---|---|---|---|---|
| Negative | | | | |
| DMSO (2% in RPMI) | 4.58 | 2.79 | 6.50 | 5.28 |
| RPMI | 4.40 | 4.51 | 11.10 | 9.38 |
| CspA (10 ug/ml) | 3.97 | 4.50 | 9.06 | 4.36 |
| Positive | | | | |
| PMA (50 ng/ml) | 99.42 | 99.04 | 97.68 | 94.14 |
| CD2/CD2R | 28.07 | 21.00 | 27.73 | 19.38 |
| (5 ul/250 ul rxn) | | | | |

Experimental

| | | | | |
|---|---|---|---|---|
| Ext. Conc. ug/ml | | | | |
| 0.012 | 19.15 | 20.61 | 21.14 | 42.21 |
| 0.024 | 21.74 | 21.39 | 24.97 | 44.04 |
| 0.049 | 25.69 | 25.73 | 23.07 | 52.94 |
| 0.098 | 31.98 | 33.96 | 31.96 | 56.78 |
| 0.195 | 34.86 | 39.65 | 31.96 | 56.78 |
| 0.390 | 37.37 | 41.89 | 38.95 | 64.69 |
| 0.780 | 43.98 | 45.57 | 46.43 | 69.48 |
| 1.560 | 43.82 | 43.76 | 51.62 | 67.34 |
| 3.125 | 45.23 | 46.00 | 53.61 | 70.47 |
| 6.250 | 47.75 | 48.57 | 58.72 | 70.90 |
| 12.50 | 52.18 | 51.58 | 66.18 | 71.30 |
| 25.00 | 58.33 | 57.66 | 66.03 | 68.57 |
| 50.00 | 59.34 | 55.05 | 76.68 | 72.85 |
| 100.00 | 67.45 | 62.05 | 82.62 | 71.63 |
| 200.00 | 67.92 | 58.43 | 85.62 | 68.54 |
| 400.00 | 64.00 | 53.22 | 86.04 | 69.96 |
| 800.00 | 47.94 | 28.16 | 89.70 | 63.01 |
| 1600 | 33.51 | 17.24 | 88.55 | 57.71 |

### Example 8

Extracts of E. purpurea flowerlets are prepared in accordance with Extraction Method 4 and concentrated to a desired level of concentration. The extracts are then tested for immunostimulatory effects using the Immune Cell Stimulation Assay Method. CD4+, CD8+, CD19+, and CD56+ stimulation results are provided in Table 8.

TABLE 8
Immunostimulatory Effects of Echinacea Angustifolia
Root Extracts Prepared Using Method 4

| | | | |
|---|---|---|---|
| CD4+ | CD8+ | CD19+ | CD56+ |

Controls

| | | | | |
|---|---|---|---|---|
| Negative | | | | |
| DMSO (2% in RPMI) | 2.000 | 5.290 | 7.370 | 7.100 |
| RPMI | 4.400 | 5.230 | 8.850 | 8.360 |
| CSPA (10 ug/ml) | 2.880 | 5.740 | 8.060 | 6.710 |
| Positive | | | | |
| PMA (50 ng/ml) | 98.770 | 98.320 | 98.820 | 94.940 |
| PHA (20 ug/ml) | 17.480 | 20.700 | 24.200 | 18.510 |
| CD2/CD2R | 40.200 | 26.600 | 37.430 | 31.460 |
| (5 ul/250 ul rxn) | | | | |

Experimental

| | | | | |
|---|---|---|---|---|
| Ext. Conc. ug/ml | | | | |
| 0.012 | 12.370 | 15.960 | 16.320 | 46.050 |
| 0.024 | 13.500 | 17.010 | 20.300 | 46.200 |
| 0.049 | 17.360 | 20.190 | 24.790 | 52.820 |
| 0.098 | 21.990 | 23.310 | 37.520 | 58.690 |
| 0.195 | 26.580 | 26.960 | 40.230 | 56.140 |
| 0.390 | 29.360 | 35.210 | 50.040 | 61.410 |
| 0.780 | 35.640 | 35.630 | 55.330 | 61.270 |
| 1.560 | 40.310 | 40.380 | 59.970 | 64.190 |
| 3.125 | 39.680 | 39.990 | 65.400 | 66.620 |
| 6.250 | 50.130 | 49.430 | 71.870 | 68.650 |
| 12.50 | 51.940 | 49.860 | 76.420 | 74.070 |
| 25.00 | 61.990 | 59.660 | 80.200 | 77.670 |
| 50.00 | 62.750 | 57.910 | 82.800 | 78.690 |
| 100.00 | 69.330 | 62.400 | 87.440 | 80.300 |
| 200.00 | 68.530 | 67.480 | 87.440 | 77.020 |
| 400.00 | 64.250 | 56.080 | 87.610 | 72.120 |
| 800.00 | 57.720 | 42.980 | 85.420 | 62.380 |
| 1600 | 47.610 | 29.880 | 80.900 | 58.750 |

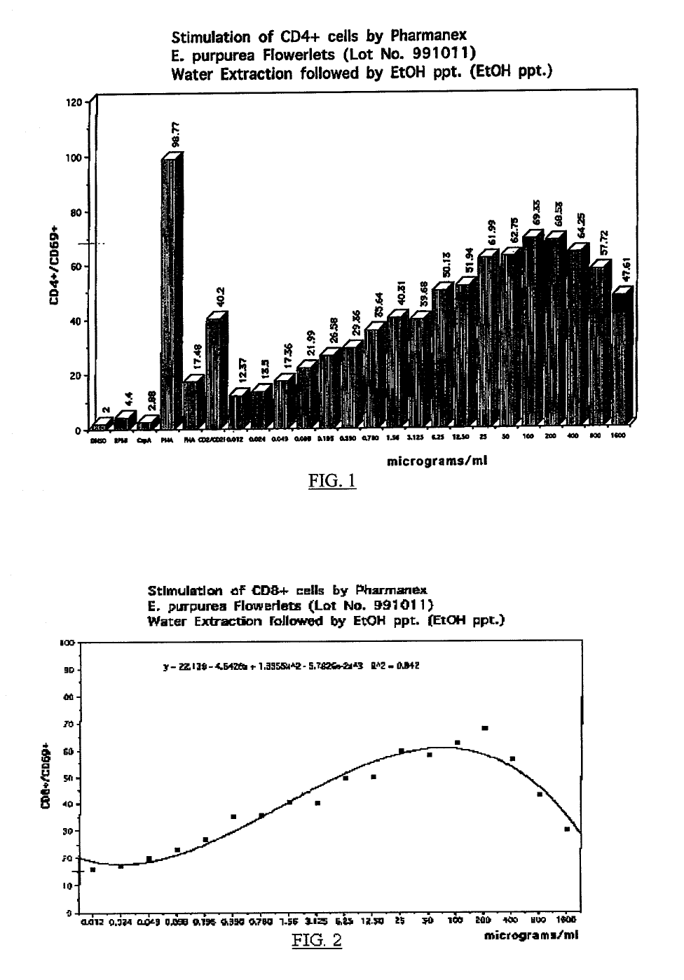

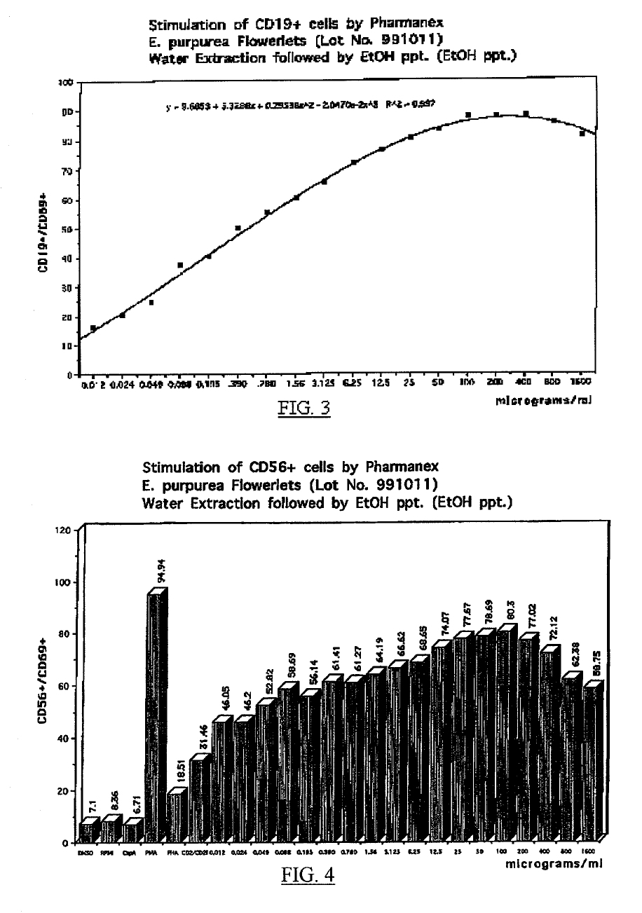

Graphical representations of the immunostimulation results contained in Table 7 above are included as FIGS. 1-4. As can be seen, immunostimulation of all immune cell types is dosage dependent. In FIG. 1, it can be seen that peak stimulation of CD4+ immune cells gradually increases with the dosage and peaks around a dosage of 100 ug/ml, declining thereafter. FIG. 2 shows that stimulation of CD8+ immune cells generally increases according to dosage amount and plateaus between about 50 and 200 ug/ml, declining thereafter. FIG. 3 shows that stimulation of CD19+ cells gradually increases according to dosage amount an plateaus between about 100 and 400 ug/ml, declining thereafter. In FIG. 4 the stimulation of CD56+ cells experiences a sharp initial increase with slight increase according to dosage increases. Stimulation generally peaks between about 25 and 100 ug/ml and declines sharply thereafter.

Utilizing the dosage response values presented in Tables 7 and 8 above, the ED50 values for each immune cell type are determined. The determination of ED50 values is well known to those skilled in the art, and a discussion thereof is found in Remmington: The Science and Practice of Pharmacy, pg. 699-702 (19^{th} ed. 1995). Extrapolated ED50 values for each immune cell are presented in Table 9.

TABLE 9
Extrapolated ED₅₀ Values for Echinacea purpurea
Flowerlets and Echinacea Angustifolia Roots

| | Immune | |
|---|---|---|
| Echinacea Source | Cell Type | ug/ml |
| E. purpurea | CD4+ | ˜0.6 |
| Flowerlets | CD8+ | ˜0.2 |
| | CD19+ | ˜0.4 |
| | CD56+ | <0.12 |
| E. angustifolia | CD4+ | ˜0.1 |
| Roots | CD8+ | ˜0.095 |
| | CD16+ | ˜0.6 |
| | CD56+ | <0.12 |

### Example 9

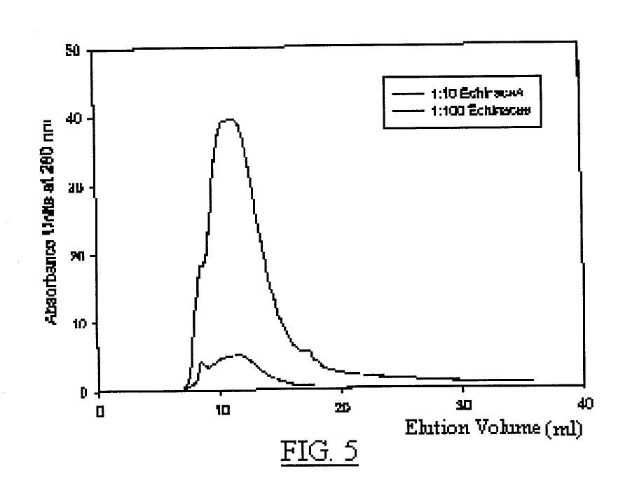

100 ul of Echinacea extract are diluted at ratios of 1:10 or 1:100 in 50 mM Tris pH 8.0 and are subjected to size exclusion chromatography using a Superdex 75 HR 10/30 column, equilibrated with 50 mM Tris pH 8.0 The column is calibrated using low molecular weight proteins (Rnase, MW 13,700; Chymotrypsinogen, MW 25,000; Ovalumin, MW 43,000; Albumin, MW 67,000) i.e. globular proteins that likely have different interactions with the column matrix as compared to oligosaccharides and polysaccharides. Elution is performed with a flow rate of 0.5 ml and 1 ml fraction were collected. A broad peak representing the active polysaccharides at OD 280 nm is observed as shown in FIG. 5 which corresponds to a molecular weight range of between 30,000 and 70,000.

It to be understood that the above-described arrangements and protocols are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method of preparing an immunostimulant composition comprising the steps of:
a) providing an Echinacea source;
b) extracting an active polysaccharide from the Echinacea source into an aqueous solution;
c) precipitating a water precipitate containing the active polysaccharide from the aqueous solution; and
d) recovering the water precipitate containing the active polysaccharide.

2. The method of claim 1, further comprising the step of:
removing the Echinacea source from the aqueous solution, prior to the step of precipitating.

3. The method of claim 1, further comprising the step of:
adding an organic solvent to the aqueous solution to remove organic materials therefrom, prior to the step of precipitating.

4. The method of claim 3, wherein the organic solvent is a water immiscible solvent.

5. The method of claim 4, wherein the water immiscible solvent is ethyl acetate.

6. The method of claim 1, further comprising the step of: reducing the solution to a lower volume, prior to the step of precipitating.

7. The method of claim 1, wherein the step of precipitating further comprises the step of: adding a precipitant to the aqueous solution to form the precipitate containing the active polysaccharide.

8. The method of claim 7, wherein the precipitant is an alcohol.

9. The method of claim 8, wherein the alcohol is a lower alkanol having from 1 to 4 carbon atoms.

10. The method of claim 9, wherein the lower alkanol is ethanol.

11. The method of claim 1, further comprising the step of:
dividing the recovered water precipitate into separate components according to size.

12. The method of claim 11, wherein the step of dividing is accomplished using size exclusion chromatography.

13. The method of claim 1, wherein the Echinacea source is a plant species selected from the group consisting of: Echinacea purpurea, Echinacea angustifolia, Echinacea pallida, and mixtures thereof.

14. The method of claim 13, wherein the Echinacea source is Echinacea purpurea.

15. The method of claim 13, wherein the Echinacea source is Echinacea angustifolia.

16. The method of claim 13, wherein the Echinacea source is Echinacea pallida.

17. The method of claim 1, wherein the Echinacea source is provided by Echinacea roots.

18. The method of claim 1, wherein the Echinacea source is provided by Echinacea aerial parts.

19. The method of claim 1, wherein the aqueous solution has a temperature from about 10° C. to about 45° C.

20. The method of claim 1, wherein the aqueous solution has a pH of from about 5 to about 7.

21. The method of claim 1, wherein the active polysaccharide comprises from about 5% to about 99% w/w of the immunostimulant composition.

22. A method of preparing an immunostimulant composition comprising the steps of:
a) providing an Echinacea source;
b) extracting an active polysaccharide from the Echinacea source into a water solution;
c) removing the Echinacea source from the water solution;
d) reducing the water solution in volume to a volume lower than an original volume;
e) adding an amount of ethanol to the water solution which is about five times greater in volume than the water solution to form a precipitate containing the active polysaccharide;
f) recovering the precipitate containing the active polysaccharide; and
g) dividing the precipitate into separate components according to size using size exclusion chromatography.
